(19) United States
(12) Patent Application Publication
(10) Pub. No.: US 2005/0036975 A1
(43) Pub. Date: Feb. 17, 2005

(54) Surfactant-free shaving aid preparation

(75) Inventors: Kerstin Heike, Hamburg (DE); Jens Treu, Norderstedt (DE); Katharina Post, Hamburg (DE); Kathrin Wolter, Hamburg (DE)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/910,202

(22) Filed: Aug. 2, 2004

(30) Foreign Application Priority Data
Aug. 1, 2003 (DE) 103 36 044.1

(51) Int. Cl. A61K 7/15; A61K 9/24

(52) U.S. Cl. 424/73

(57) ABSTRACT

The present invention is a shaving aid preparation suitable for electric shavers comprising 0.05 to 0.5% by weight of cross-linked polyacrylate, 1 to 7% by weight of glycerol, 0.1 to 1% by weight of xanthan gum, and water, wherein the preparation has a viscosity of 500 to 5000 mPa·s, and wherein the preparation is free of surfactants, in particular free of sarcosinates. The present invention also includes a method of shaving comprising applying the shaving aid preparation and a shaver comprising the preparation.

### FIELD OF THE INVENTION

The present invention relates to preparations which are tailor-made for special shavers. The invention at the same time includes lipid-containing preparations, for example preparations present as emulsions or gel creams, and lipid-free preparations, for example preparations present as gels. Lipid-containing preparations can also be free from emulsifiers and are then designated as gel creams. It is common to all these preparations, however, that they can be formulated free from surfactants, in particular free from sarcosinates.

### BACKGROUND OF THE INVENTION

Shaving is used for the mechanical removal of hair from the skin by cutting off the beard. There are two known processes here: wet- or dry-shaving. In the case of wet-shaving, the hair is first made soft and flexible by application of a soap-containing shaving aid preparation or a soap-containing foam. Subsequently to this, the hair is cut off using a blade. In the course of this, the blade glides over the skin and irritates the latter. This skin irritation can be counteracted by choosing shaving aid preparations which allow particularly easy gliding of the blade over the skin. Another possibility is the dry shave. In the case of the dry shave, a blade does not glide directly over the skin, but a “cutting foil”, behind which is concealed the blade. The blade is electrically driven and by means of its movements cuts off hairs which pass through the cutting foil. In this way, the blade does not come into direct contact with the skin. If preparations are used which are intended to improve the shaving result, these should make the hair stiff so that it can pass through the cutting foil readily. This would be achieved, for example, by preparations containing alcohol, which extract moisture from the hair and thereby make it particularly hard.

If an electric dry shaver is used with a customary shaving aid preparation for wet shaving, an unsatisfactory result is obtained.

A shaver such as is described in European Patent Application WO 98/08659 represents an intermediate form between wet and dry shavers. Like a conventional dry shaver, it has a cutting foil, but a shaving aid preparation can additionally be added so that during shaving the skin is brought into contact with the shaving aid preparation. By this means, the shaver glides particularly well over the skin if the shaving aid preparation takes this fact into account. Shaving aid preparations of this type can be present as an emulsion (lipid- and emulsifier-containing), gel cream (lipid-containing and emulsifier-free) or as a gel (lipid- and emulsifier-free). In order to obtain a good result, what matters, among other things, is that the glide film is not too thick or too viscous, and foam formation, which increases the distance between the skin and shaver, does not occur. A further problem is the gumming up of the shaver itself after completion of the shave. If the shaver is not regularly rinsed with water, a deposit of beard stubble and shaving aid preparation is formed in the hair chamber of the shaver and on blades and cutting foil. Moreover, the problem of corrosion occurs, since, in contrast to dry shavers, the blade comes into contact with moisture and thereby corrodes. If a corrosion-preventing shaving aid preparation could be made available, the user, for example, could carry out the shave under the shower.

In summary, this means that the four main requirements (high glideability, no gumming up of the cutting foil, no corrosion, no skin irritation) have to be fulfilled simultaneously in a product of this type, but the aids customarily employed for fulfilling individual requirements in each case make another requirement unfulfillable.

Additionally, the shaving aid preparation should be adapted to the skin of the person to be shaved: a lipid-free shaving aid preparation, which, for example, can be present as a gel, is particularly suitable for greasy skin or for skin which is prone to acne formation. At the same time, however, it must be ensured that an adequate glideability of the shaver on the skin can be achieved, since this is customarily brought about by the oil content.

An oil- or lipid-containing shaving aid preparation, which can be present, for example, as an emulsion or gel cream, is used, however, for the care of the skin and is particularly suitable for dry skin. Customary oil-containing shaving aid preparations used for the dry shave have either greasy or moist haptics. Both forms are unsuitable for an electric shave within the meaning of the invention. Although the former lubricate the skin well, such that adequate gliding of the shaver on the skin is achieved, they form, however, a layer on the skin, whereby the hair length remaining after the shave increases and thus a poor shave result is achieved. On the other hand, moist oil-containing shaving aid preparations of the prior art are absorbed rapidly into the skin and thus do not adequately permanently make available the necessary glideability.

For mixed skin, it is advantageous if a certain oil or lipid content is indeed present in the shaving aid preparation, but this is free from emulsifiers and can be present, for example, as a gel cream. The presence of surfactants or emulsifiers is in each case unsuitable, since these ingredients frequently lead to skin irritation.

The specification WO 2002/87519 discloses shaving preparations which contain water, a cleansing or conditioning agent for skin or hair and at least one water-soluble polymer and the shear stress ratio (normal stress/shear stress) is greater than 3, measured at a shear rate of 800 s⁻¹. Nothing is reported about preparations which contain no surfactants.

The specification US 2003/0026775 discloses shaving preparations which contain water, a foam-forming surfactant and a volatile foaming agent and have specific rheological properties. Nothing is reported about preparations which contain no surfactants.

DE 10057925 discloses preparations, in particular shaving gels, which are suitable for shaving by means of a mechanical shaver, in particular an electric shaver, and which contain one or more N-acylsarcosinates, one or more polysaccharides, one or more substances which form a gel structure in water, which are not polysaccharides and which advantageously have surface-active properties, one or more solubilizers, preferably chosen from the group consisting of the polyethoxylated saturated fatty or oil components and water. Nothing is reported about preparations which contain no surfactants.

All these preparations are indeed suitable for an electric or dry shave, but regularly lead to skin irritation, at least in some users.

### SUMMARY OF THE INVENTION

The present invention was based on the object of finding preparations which are particularly skin-friendly. It has turned out as unpredictable for the person skilled in the art that lipid-containing, at the same time emulsifier-containing or emulsifier-free or else lipid-free and emulsifier-free shaving aid preparations suitable for electric shavers comprising
0.05 to 0.5% by weight of crosslinked polyacrylates,
1 to 7% by weight of glycerol,
0.1 to 1% by weight of xanthan gum
and water,
where the preparation has a viscosity of 500 to 5000 mPa·s, measured using the Haake viscometer ‘Viscotester VT02’ measured at room temperature in a vessel having a diameter of 42 mm and reading off the measurement after 30 s, characterized in that the preparation is free of surfactants, in particular free of sarcosinates,
   remedy the deficiencies of the prior art.

### DETAILED DESCRIPTION OF THE PREFERRED EMBODIMENTS

Shaving aid preparations according to the invention are distinguished by an extremely mild, good glideability of the shaver on the skin surface wetted with shaving aid preparation and good caring properties. At the same time, such preparations feel fresh and cooling on the skin and moisturize the skin well. Such preparations can be present as a gel, lipid-containing gel (gel cream) or as a cream. In the case of a gel, the advantage of such preparations in particular consists in the fact that the glideability of the shaver on the skin is considerably improved without oils additionally having to be incorporated. Thus, these preparations are particularly suitable for greasy skin or skin prone to acne. At the same time, these preparations can now be made available surfactant-free. In the case of a lipid-containing gel (gel cream), the advantage of such preparations consists in particular in the fact that less lipid, no emulsifier and no surfactant have to be employed and the preparation nevertheless makes possible adequate gliding of the shaver over the skin. Such preparations are in particular suitable for “mixed skin”. In the case of a cream, the advantage of such preparations in particular consists in the fact that lipids caring for the skin and no surfactant can be employed and the preparation thus makes possible care of the skin during shaving.

It has further been found that it is preferred in the case of lipid-containing preparations if polyethylene glycols are additionally present. Particularly preferably, the polyethylene glycol(s) is/are chosen from the group consisting of PEG 20, 30, 45 and 90. PEG 45 is very particularly preferred. As a result of the incorporation of polyethylene glycols, a better gliding power of the shaver over the skin can be achieved.

It is particularly preferred if the content of lipids and/or oils is 1 to 5% by weight. By this means an optimum care capacity and sensory function is achieved.

Furthermore, it is preferred if in such shaving aid preparations present as an O/W emulsion the content of the emulsifiers is 0.5 to 3%. Different lipid contents and viscosities can thus be stably formulated.

Preferably, the lipids are chosen from the group consisting of the esters of saturated or unsaturated, branched or unbranched alkanecarboxylic acids of a chain length of 3 to 30 C atoms and saturated or unsaturated, branched or unbranched alcohols of a chain length of 3 to 30 C atoms, particularly preferably ethyl hexyl cocoate.

It is furthermore preferred if, in such shaving aid preparations present as an O/W emulsion, the emulsifiers are chosen from the group consisting of the mono-, di- and trialkyl phosphoric acid esters and their ethoxylates, particularly preferably triceteareth 4 phosphate.

In the case of lipid-free and emulsifier-free shaving aid preparations present as a gel, it is particularly preferred if a content of PEG-40-hydrogenated castor oil fatty acid ethers of 0.75 to 2% by weight is additionally present.

In all these lipid-free shaving aid preparations, it is particularly preferred if a content of cellulose derivatives, preferably hydroxyethylcellulose in concentrations of 0.1 to 1% by weight, preferably 0.1 to 0.6% by weight, particularly preferably 0.2% by weight, is additionally present.

The invention furthermore comprises the use of such shaving aid preparations as an aid in electric shaving, and a shaver comprising such shaving aid preparations.

The leaving out of an individual constituent adversely affects the unique properties of the overall composition. Therefore, all specified constituents of the shaving aid preparations according to the invention are absolutely necessary in order to carry out the invention.

Presence of xanthan gum is also advantageous. Xanthan gum (CAS No. 11138-66-2) is an anionic heteropolysaccharide which as a rule is formed by fermentation of maize sugar and is isolated as the potassium salt. It is produced by Xanthomonas campestris and some other species under aerobic conditions with a molecular weight of 2×10⁶ to 24×10⁶. Xanthan is formed of a chain containing β-1,4-bonded glucose (cellulose) having side chains. The structure of the subgroups consists of glucose, mannose, glucuronic acid, acetate and pyruvate. Xanthan is the name for the first microbial anionic heteropolysaccharide. It is produced by Xanthomonas campestris and some other species under aerobic conditions with a molecular weight of 2-15×10⁶. Xanthan is formed of a chain containing β-1,4-bonded glucose (cellulose) having side chains. The structure of the subgroups consists of glucose, mannose, glucuronic acid, acetate and pyruvate. The number of pyruvate units determines the viscosity of the xanthan. Xanthan is produced in two-day batch cultures with a yield of 70-90%, based on carbohydrate employed. Yields of 25-30 g/l are achieved here. Working up is carried out after destruction of the culture by precipitation with, for example, 2-propanol. Xanthan is subsequently dried and ground. Xanthan is characterized by the structure 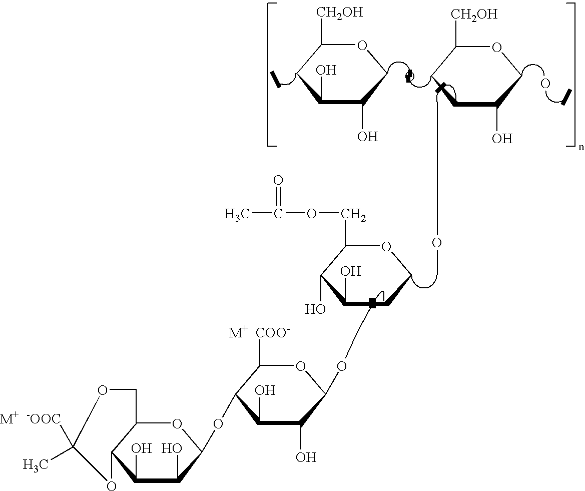
where M⁺ can be Na⁺, K⁺ or half an equivalent of Ca²⁺.

According to the invention, the preparations advantageously contain 0.01 to 10% by weight, particularly advantageously 0.01 to 5% by weight, very particularly preferably 0.5 to 3% by weight, of one or more polysaccharides, in each case based on the total weight of the preparations.

Crosslinked polyacrylates within the meaning of the invention are acrylate-alkyl acrylate copolymers, in particular those which are chosen from the group consisting of the ‘carbomers or carbopols’ (Carbopol® is actually a registered trademark of the B. F. Goodrich Company). In particular, the acrylate-alkyl acrylate copolymer(s) advantageous according to the invention is/are distinguished by the following structure:

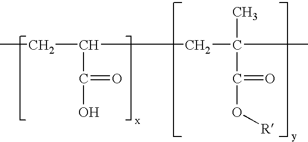

In this structure, R′ is a long-chain alkyl radical and x and y are numbers which symbolize the respective stoichiometric proportion of the respective comonomers.

Copolymers of C₁₀₋₃₀-alkyl acrylates and one or more monomers of acrylic acid, methacrylic acid or their esters which are crosslinked with an allyl ether of sucrose or an allyl ether of pentaerythritol are furthermore advantageous.

The following polyacrylate types are particularly advantageous according to the invention: Carbomer 907 (molecular wt. 450 000), Carbomer 910 (molecular wt. 750 000), Carbomer 941 and Carbomer 951 (molecular wt. 1 250 000), Carbomer 934, Carbomer 940 and Carbomer 954 (molecular wt. 3 000 000), Carbomer 940 (molecular wt. 4 000 000), Carbomer 980 (molecular wt. 4 000 000), Carbomer 981 (molecular wt. 1 250 000), Carbomer 984 (molecular wt. 3 000 000) (Carbomer 980, 981 and 984 are acrylic acid polymers which are polymerized in a mixture of ethyl acetate and cyclohexane), Carbomer 974 P (molecular wt. 3 000 000), Carbomer 1342 (molecular wt. 1 300 000).

Carbomer 1342 has a similar composition to the Pemulen types. Pemulen is the trade name for a copolymer of C₁₀₋₃₀-alkyl acrylates and one or more monomers of acrylic acid, methacrylic acid or their esters, which are crosslinked with an allyl ether of sucrose or an allyl ether of pentaerythritol (CTFA name: Acrylates/C₁₀₋₃₀ Alkyl Acrylate Crosspolymer). Pemulens are high molecular weight polymers having a high hydrophilic content and at the same time having a low content of lipophilic components.

Carbomer 1342 is the substance preferred according to the invention, which forms a gel structure in water.

The water content of the preparations according to the invention is as a rule between 70 and 98% by weight, based on the total weight of the preparations, in particular between 85 and 95% by weight.

Cellulose derivatives within the meaning of the invention are cellulose ethers, for example methyl- or hydroxyethylcelluloses. They are distinguished by the following structural formula 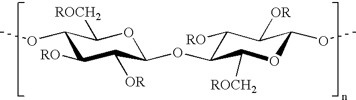
in which R can be a hydrogen, alkyl or a hydroxyalkyl group.

Particularly advantageous within the meaning of the present invention are the cellulose mixed ethers, which contain methyl, 2-hydroxyethyl, 2-hydroxypropyl or 2-hydroxybutyl groups next to one another. (Hydroxypropyl)methylcelluloses are particularly preferred, for example those obtainable from the Dow Chemical Corp. under the trade name Methocel E4M.

Furthermore advantageous according to the invention is sodium carboxymethylcellulose, the sodium salt of the glycolic acid ether of cellulose, for which R in structural formula I can be a hydrogen or CH₂—COONa. The sodium carboxymethylcellulose, also called cellulose gum, obtainable under the trade name Natrosol Plus 330 CS from Aqualon is particularly preferred.

Hydroxyethylcellulose is very particularly preferred.

Advantageous polar oil components can further be chosen within the meaning of the present invention from the group consisting of the esters of saturated or unsaturated, branched or unbranched alkanecarboxylic acids of a chain length of 3 to 30 C atoms and saturated or unsaturated, branched or unbranched alcohols of a chain length of 3 to 30 C atoms, and from the group consisting of the esters of aromatic carboxylic acids and saturated or unsaturated, branched or unbranched alcohols of a chain length of 3 to 30 C atoms. Such ester oils can then advantageously be chosen from the group consisting of octyl palmitate, ethylhexyl cocoate, octyl isostearate, octyldodecyl myristate, octyldodecanol, cetearyl isononanoate, isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, stearyl heptanoate, oleyl oleate, oleyl erucate, erucyl oleate, erucyl erucate, tridecyl stearate, tridecyl trimellitate, and synthetic, semi-synthetic and natural mixtures of such esters.

Furthermore, the oil phase can advantageously be chosen from the group consisting of the dialkyl ethers and dialkyl carbonates.

Furthermore, the oil phase can likewise advantageously also contain non-polar oils, for example those which are chosen from the group consisting of the branched and unbranched hydrocarbons and hydrocarbon waxes, in particular mineral oil, petroleum jelly (petrolatum), paraffin oil, squalane and squalene, polyolefins, hydrogenated polyisobutenes and isohexadecane. Among the polyolefins, polydecenes and hydrogenated polyisobutenes are the preferred substances.

The non-polar oil components can advantageously be present in a concentration of up to 60% by weight based on the total weight of the lipid phase in the preparations according to the invention.

The preparations according to the invention, which can be present in the form of an emulsion, contain one or more emulsifiers. These emulsifiers can advantageously be chosen from the group consisting of the non-ionic, anionic, cationic or amphoteric emulsifiers.

Amongst the non-ionic emulsifiers are

a) partial fatty acid esters and fatty acid esters of polyhydric alcohols and their ethoxylated derivatives (e.g. glyceryl monostearate, sorbitan stearate, glycerylstearyl citrate, sucrose stearate)
b) ethoxylated fatty alcohols and fatty acids
c) ethoxylated fatty amines, fatty acid amides, fatty acid alkanolamides
d) alkylphenol polyglycol ether (e.g. Triton X)
e) sugar derivatives (esters or ethers of glucose, sucrose and other sugars; e.g. alkylpolyglycosides such as polyglyceryl 3-methylglucose distearate, methylglucose sesquistearate)
   Amongst the anionic emulsifiers are
a) soaps (e.g. sodium stearate)
b) fatty alcohol sulphates
c) mono-, di- and trialkylphosphoric acid esters and their ethoxylates such as, for example, triceteareth-4 phosphate.
   Amongst the cationic emulsifiers are
a) quaternary ammonium compounds having a long-chain aliphatic radical, e.g. distearyldimonium chloride
   Amongst the amphoteric emulsifiers are
a) alkylaminoalkanecarboxylic acids
b) betaines, sulfobetaines
c) imidazoline derivatives

Furthermore, there are naturally occurring emulsifiers, which include beeswax, wool wax, lecithin and sterols.

O/W emulsifiers can, for example, be advantageously chosen from the group consisting of the polyethoxylated or polypropoxylated or polyethoxylated and polypropoxylated products, e.g.:

the fatty alcohol ethoxylates
the ethoxylated wool wax alcohols,
the polyethylene glycol ethers of the general formula R—O—(—CH₂—CH₂—O—)ₙ—R′,
the fatty acid ethoxylates of the general formula R—COO—(—CH₂—CH₂—O—)ₙ—H,
the etherified fatty acid ethoxylates of the general formula R—COO—(—CH₂—CH₂—O—)ₙ—R′,
the esterified fatty acid ethoxylates of the general formula R—COO—(—CH₂—CH₂—O—)ₙ—C(O)—R′,
the polyethylene glycol glycerol fatty acid esters
the ethoxylated sorbitan esters
the cholesterol ethoxylates
the ethoxylated triglycerides
the alkyl ether carboxylic acids of the general formula R—O—(—CH₂—CH₂—O—)ₙ—CH₂—COOH in which n is a number from 5 to 30,
the polyoxyethylene sorbitol fatty acid esters,
the alkyl ether sulphates of the general formula R—O—(—CH₂—CH₂—O—)ₙ—SO₃—H
the fatty alcohol propoxylates of the general formula R—O—(—CH₂—CH(CH₃)—O—)ₙ—H,
the polypropylene glycol ethers of the general formula R—O—(—CH₂—CH(CH₃)—O—)ₙ—R′,
the propoxylated wool wax alcohols,
the etherified fatty acid propoxylates R—COO—(—CH₂—CH(CH₃)—O—)ₙ—R′,
the esterified fatty acid propoxylates of the general formula R—COO—(—CH₂—CH(CH₃)—O—)ₙ—C(O)—R′,
the fatty acid propoxylates of the general formula R—COO—(—CH₂—CH(CH₃)—O—)ₙ—H,
the polypropylene glycol glycerol fatty acid esters
the propoxylated sorbitan esters
the cholesterol propoxylates
the propoxylated triglycerides
the alkyl ether carboxylic acids of the general formula R—O—(—CH₂—CH(CH₃)O—)ₙ—CH₂—COOH
the alkyl ether sulphates or the acids on which these sulphates are based of the general formula R—O—(—CH₂—CH(CH₃)—O—)ₙ—SO₃—H
the fatty alcohol ethoxylates/propoxylates of the general formula R—O—Xₙ—Yₘ—H,
the polypropylene glycol ethers of the general formula R—O—Xₙ—Yₘ—R′,
the etherified fatty acid propoxylates of the general formula R—COO—Xₙ—Yₘ—R′,
the fatty acid ethoxylates/propoxylates of the general formula R—COO—Xₙ—Yₘ—H.

According to the invention, the polyethoxylated or polypropoxylated or polyethoxylated and polypropoxylated O/W emulsifiers are advantageously chosen from the group of substances having HLBs of 11-18, very particularly advantageously having HLBs of 14.5-15.5, provided the O/W emulsifiers contain saturated radicals R and R′. If the O/W emulsifiers contain unsaturated radicals R and/or R′, or isoalkyl derivatives are present, the preferred HLB of such emulsifiers can also be lower or higher.

Polyethylene glycol (13) cetylstearyl ether (Ceteareth-13), polyethylene glycol (14) cetylstearyl ether (Ceteareth-14), polyethylene glycol (15) cetylstearyl ether (Ceteareth-15), polyethylene glycol (16) cetylstearyl ether (Ceteareth-16), polyethylene glycol (17) cetylstearyl ether (Ceteareth-17), polyethylene glycol (18) cetylstearyl ether (Ceteareth-18), polyethylene glycol (19) cetylstearyl ether (Ceteareth-19), polyethylene glycol (20) cetylstearyl ether (Ceteareth-20).

In addition, it is convenient according to the invention to employ PEG derivatives in order positively to influence the gliding power of the shaver on the skin. Within the meaning of the invention, in this case especially long-chain PEG derivatives such as PEG 20, 30, 45 and 90 appear particularly suitable.

The viscosity within the meaning of the invention is measured using the Haake viscometer ‘Viscotester VT02’ at room temperature in a vessel having a diameter of 42 mm and reading off the measurement after 30 s.

The cosmetic preparations according to the invention can contain cosmetic excipients such as are customarily used in such preparations, e.g. preservatives, bactericides, antioxidants, perfumes, agents for preventing foaming, colourants, pigments which have a colouring action, thickening agents, surface-active substances, emulsifiers, plasticizing substances, moisturizing and/or humectant substances, fats, oils, waxes or other customary constituents of a cosmetic formulation such as alcohols, polyols, polymers, foam stabilizers, electrolytes, pH-regulating substances, organic solvents or silicone derivatives.

It may furthermore be advantageous according to the invention to add complexing agents to the preparations.

Complexing agents are excipients of cosmetology or of medicinal pharmaceutics which are known per se. By the complexation of troublesome metals such as Mn, Fe, Cu and others, it is possible, for example, to prevent undesired chemical reactions in cosmetic or dermatological preparations.

Complexing agents, in particular chelators, form complexes with metal atoms, which in the presence of one or more polybasic complexing agents, that is chelators, are metallacycles. Chelates are compounds in which an individual ligand occupies more than one coordination site on a central atom. In this case, normally linear compounds are thus closed via a metal atom or ion to give rings. The number of ligands bound depends on the coordination number of the central metal. The prerequisite for chelate formation is that the compound reacting with the metal contains two or more atomic groups which act as electron donors.

The complexing agent(s) can advantageously be chosen from the group consisting of the customary compounds, preferably at least one substance being chosen from the group consisting of tartaric acid and its anions, citric acid and its anions, aminopolycarboxylic acids and their anions (such as, for example, ethylenediaminetetraacetic acid (EDTA) and its anions, nitrilotriacetic acid (NTA) and its anions, hydroxyethylenediaminotriacetic acid (HOEDTA) and its anions, diethyleneaminopentaacetic acid (DPTA) and its anions, trans-1,2-diaminocyclohexanetetraacetic acid (CDTA) and its anions) and tetrasodium iminodisuccinate.

According to the invention, the complexing agent(s) is/are advantageously present in cosmetic or dermatological preparations preferably to 0.01% by weight to 10% by weight, preferably to 0.05% by weight to 5% by weight, particularly preferably to 0.1-2.0% by weight, based on the total weight of the preparations.

It is possible in all of this in the individual case that the aforementioned concentration data are slightly exceeded or fallen short of and nevertheless shaving aid preparations according to the invention are obtained. In view of the widespread variety of suitable components of shaving aid preparations of this type, this is not unexpected for the person skilled in the art, so that he knows that in the case of such an exceeding or falling short, the base of the invention is not left.

The following examples are intended to illustrate the present invention without restricting it. The numerical values in the examples denote percentages by weight, based on the total weight of the respective preparations.

### EXAMPLES

Emulsions

| | A | B | C | D |
|---|---|---|---|---|
| | m [%] | m [%] | m [%] | m [%] |
| Acrylates/C10-30 Alkyl Acrylate | 0.5000 | 0.3000 | 0.0500 | 0.175 |
| Crosspolymer | | | | |
| Ethylhexyl Cocoate | 1.0000 | 0.7550 | 0.5000 | 1.0000 |
| Biosaccharide gum | 3.0000 | 2.0000 | 1.0000 | 2.0000 |
| Glycerol | 5.0000 | 4.0000 | 2.0000 | 3.0000 |
| Isohexadecane | 4.0000 | 3.0000 | 1.0000 | 2.0000 |
| PEG-45 M | 0.5000 | 1.0000 | 2.0000 | 0.7500 |
| Sodium Hydroxide | 0.1000 | 0.0750 | 0.1500 | 0.1250 |
| Triceteareth-4 Phosphate | 1.5000 | 1.0000 | 0.5000 | 0.7000 |
| Xanthan Gum | 0.2000 | 0.3000 | 0.5000 | 0.2000 |
| Fragrance | 0.0500 | 0.0500 | 0.0500 | 0.0500 |
| Water | to 100 | to 100 | to 100 | to 100 |

Gels

| | A | B | C | D |
|---|---|---|---|---|
| | m [%] | m [%] | m [%] | m [%] |
| Acrylates/C10-30 Alkyl Acrylate | 0.5000 | 0.1500 | 0.2000 | 0.0500 |
| Crosspolymer | | | | |
| Fragrance | 0.2000 | 0.2000 | 0.2000 | 0.2000 |
| Glycerol | 7.0000 | 5.0000 | 4.0000 | 3.0000 |
| Hydroxyethylcellulose | 0.1000 | 0.2000 | 0.4000 | 0.6000 |
| Methylparaben | 0.2000 | 0.2000 | 0.2000 | 0.2000 |
| PEG-40 Hydrogenated Castor Oil | 2.0000 | 1.0000 | 1.5000 | 0.7500 |
| Sodium Hydroxide | 0.1500 | 0.1000 | 0.1000 | 0.0750 |
| Xanthan Gum | 0.1000 | 0.2500 | 0.3000 | 0.5000 |
| Water | to 100 | to 100 | to 100 | to 100 |

Gel creams

| | A | B | C | D |
|---|---|---|---|---|
| | m [%] | m [%] | m [%] | m [%] |
| Acrylates/C10-30 Alkyl Acrylate | 0.5000 | 0.1500 | 0.2500 | 0.0750 |
| Crosspolymer | | | | |
| Glycerol | 5.0000 | 3.0000 | 2.0000 | 1.0000 |
| Isohexadecane | 5.0000 | 2.0000 | 2.5000 | 1.0000 |
| PEG-45 M | 0.5000 | 0.7500 | 2.5000 | 5.0000 |
| Sodium Hydroxide | 0.1000 | 0.1000 | 1.0000 | 2.5000 |
| Fragrance | 0.0500 | 0.0500 | 0.0500 | 0.0500 |
| Xanthan Gum | 1.0000 | 0.2000 | 0.5000 | 0.1000 |
| Water | to 100 | to 100 | to 100 | to 100 |

1. A shaving aid preparation suitable for electric shavers, comprising
0.05 to 0.5% by weight of at least one cross-linked polyacrylate,
1 to 7% by weight of glycerol,
0.1 to 1% by weight of xanthan gum, and
water,
wherein the preparation has a viscosity of 500 to 5,000 mPa·s, and
wherein the preparation is free of sarcinosates.

2. The preparation as claimed in claim 1, further comprising at least one lipid.

3. The preparation as claimed in claim 2, wherein the at least one lipid is present in an amount from 1 to 5% by weight.

4. The preparation as claimed in claim 2, further comprising at least one polyethylene glycol.

5. The preparation as claimed in claim 4, wherein the at least one polyethylene glycol includes at least one polyethylene glycol selected from the group consisting of PEG 20, 30, 45 and 90.

6. The preparation as claimed in claim 5, wherein the at least one polyethylene glycol includes PEG-45.

7. The preparation as claimed in claim 1, further comprising at least one lipid and 0.5 to 3% by weight of at least one emulsifier, and wherein the preparation is an oil-in-water emulsion.

8. The preparation as claimed in claim 1, further comprising at least one lipid selected from the group consisting of the esters of saturated or unsaturated, branched or unbranched alkanecarboxylic acids of a chain length of 3 to 30 C atoms and saturated or unsaturated, branched or unbranched alcohols of a chain length of 3 to 30 C atoms.

9. The preparation as claimed in claim 8, wherein the at least one lipid includes ethyl hexyl cocoate.

10. The preparation as claimed in claim 1, further comprising at least one lipid and further comprising at least one emulsifier selected from the group consisting of the mono-, di- and trialkylphosphoric acid esters and their ethoxylates, wherein the preparation is an oil-in-water emulsion.

11. The preparation as claimed in claim 10, wherein the at least one emulsifier includes triceteareth-4 phosphate.

12. The preparation as claimed in claim 1, further comprising 0.75 to 2% of a PEG-40-hydrogenated castor oil fatty acid ether, wherein the preparation is a gel and wherein the preparation is free of emulsifiers.

13. The preparation as claimed in claim 1, further comprising at least one lipid and further comprising 0.1 to 1% by weight of at least one cellulose derivative.

14. The preparation as claimed in claim 13, wherein the at least one cellulose derivative includes at least one cellulose derivative selected from the group consisting of hydroxymethylcelluloses and sodium carboxymethylcellulose.

15. The preparation as claimed in claim 13, wherein the at least one cellulose derivative includes hydroxyethyl-cellulose.

16. The preparation as claimed in claim 13, wherein the at least one cellulose derivative is present in a concentration of from 0.1 to 0.6% by weight.

17. The preparation as claimed in claim 13, wherein the at least one cellulose derivative is present in a concentration of 0.2% by weight.

18. A shaving aid preparation suitable for electric shavers comprising
(a) 0.05 to 0.5% by weight of at least one cross-linked polyacrylate,
(b) 1 to 7% by weight of glycerol,
(c) 0.1 to 1% by weight of xanthan gum, and
(d) water,
wherein the preparation has a viscosity of 500 to 5,000 mPa·s, and
wherein the preparation is free of surfactants.

19. The preparation as claimed in claim 18, further comprising at least one lipid.

20. The preparation as claimed in claim 19, wherein the at least one lipid is present in an amount from 1 to 5% by weight.

21. The preparation as claimed in claim 19, further comprising at least one polyethylene glycol.

22. The preparation as claimed in claim 21, wherein the at least one polyethylene glycol includes at least one polyethylene glycol selected from the group consisting of PEG 20, 30, 45 and 90.

23. The preparation as claimed in claim 22, wherein the at least one polyethylene glycol includes PEG-45.

24. The preparation as claimed in claim 18, further comprising at least one lipid selected from the group consisting of the esters of saturated or unsaturated, branched or unbranched alkanecarboxylic acids of a chain length of 3 to 30 C atoms and saturated or unsaturated, branched or unbranched alcohols of a chain length of 3 to 30 C atoms.

25. The preparation as claimed in claim 24, wherein the at least one lipid includes ethyl hexyl cocoate.

26. The preparation as claimed in claim 18, further comprising 0.75 to 2% of a PEG-40-hydrogenated castor oil fatty acid ether, said preparation being a gel.

27. The preparation as claimed in claim 18, further comprising at least one lipid and further comprising 0.1 to 1% by weight of at least one cellulose derivative.

28. The preparation as claimed in claim 27, wherein the at least one cellulose derivative includes at least one selected from the group consisting of hydroxymethylcelluloses and sodium carboxymethylcellulose.

29. The preparation as claimed in claim 27, wherein the at least one cellulose derivative includes hydroxyethyl-cellulose.

30. The preparation as claimed in claim 27, wherein the at least one cellulose derivative is present in a concentration of from 0.1 to 0.6% by weight.

31. The preparation as claimed in claim 27, wherein the at least one cellulose derivative is present in a concentration of 0.2% by weight.

32. A method of shaving comprising
applying to the area to be shaved a shaving aid preparation comprising
0.05 to 0.5% by weight of at least one cross-linked polyacrylate,
1 to 7% by weight of glycerol,
0.1 to 1% by weight of xanthan gum, and
water,
wherein the preparation has a viscosity of 500 to 5,000 mPa·s and
wherein the preparation is free of sarcosinates, and
shaving the area to be shaved with and electric shaver.

33. The method as claimed in claim 32, wherein the preparation is free of surfactants.

34. The method as claimed in claim 32, wherein the preparation further comprises at least one lipid.

35. The method as claimed in claim 32, wherein the preparation further comprises at least one polyethylene glycol.

36. The method as claimed in claim 35, wherein the at least one polyethylene glycol includes at least one polyethylene glycol selected from the group consisting of PEG 20, 30, 45 and 90.

37. The method as claimed in claim 32, wherein the preparation further comprises at least one lipid and 0.5 to 3% by weight of at least one emulsifier, and wherein the preparation is an oil-in-water emulsion.

38. The method as claimed in claim 32, wherein the preparation further comprises at least one lipid selected from the group consisting of the esters of saturated or unsaturated, branched or unbranched alkanecarboxylic acids of a chain length of 3 to 30 C atoms and saturated or unsaturated, branched or unbranched alcohols of a chain length of 3 to 30 C atoms.

39. The method as claimed in claim 38, wherein the at least one lipid includes ethyl hexyl cocoate.

40. The method as claimed in claim 32, wherein the preparation further comprises at least one lipid and further comprising at least one emulsifier selected from the group consisting of the mono-, di- and trialkylphosphoric acid esters and their ethoxylates, wherein the preparation is an oil-in-water emulsion.

41. The method as claimed in claim 40, wherein the at least one emulsifier includes triceteareth-4 phosphate.

42. The method as claimed in claim 32, wherein the preparation further comprises 0.75 to 2% of a PEG-40-hydrogenated castor oil fatty acid ether, wherein the preparation is a gel, and wherein the preparation is free of emulsifiers.

43. The method as claimed in claim 32, wherein the preparation further comprises at least one lipid and further comprising 0.1 to 1% by weight of at least one cellulose derivative.

44. The method as claimed in claim 43, wherein the at least one cellulose derivative includes at least one selected from the group consisting of hydroxymethylcelluloses and sodium carboxymethylcellulose.

45. The method as claimed in claim 43, wherein the at least one cellulose derivative includes hydroxyethyl-cellulose.

46. A shaver comprising a shaving aid preparation comprising
0.05 to 0.5% by weight of at least one cross-linked polyacrylate,
1 to 7% by weight of glycerol,
0.1 to 1% by weight of xanthan gum, and
water,
wherein the preparation has a viscosity of 500 to 5,000 mPa·s and
wherein the preparation is free of sarcosinates.

47. The shaver as claimed in claim 46, wherein the preparation is free of surfactants.

48. The shaver as claimed in claim 46, wherein the preparation further comprises at least one lipid.

49. The shaver as claimed in claim 48, wherein the preparation further comprises at least one polyethylene glycol.

50. The shaver as claimed in claim 49, wherein the at least one polyethylene glycol includes at least one polyethylene glycol selected from the group consisting of PEG 20, 30, 45 and 90.

51. The shaver as claimed in claim 46, wherein the preparation further comprises at least one lipid and 0.5 to 3% by weight of at least one emulsifier, and wherein the preparation is an oil-in-water emulsion.

52. The shaver as claimed in claim 46, wherein the preparation further comprises at least one lipid selected from the group consisting of the esters of saturated or unsaturated, branched or unbranched alkanecarboxylic acids of a chain length of 3 to 30 C atoms and saturated or unsaturated, branched or unbranched alcohols of a chain length of 3 to 30 C atoms.

53. The shaver as claimed in claim 52, wherein the at least one lipid includes ethyl hexyl cocoate.

54. The shaver as claimed in claim 46, wherein the preparation further comprises at least one lipid and further comprising at least one emulsifier selected from the group consisting of the mono-, di- and trialkylphosphoric acid esters and their ethoxylates, wherein the preparation is an oil-in-water emulsion.

55. The shaver as claimed in claim 54, wherein the at least one emulsifier includes triceteareth-4 phosphate.

56. The shaver as claimed in claim 46, wherein the preparation further comprises 0.75 to 2% of a PEG-40-hydrogenated castor oil fatty acid ether, wherein the preparation is a gel, and wherein the preparation is free of emulsifiers.

57. The shaver as claimed in claim 46, wherein the preparation further comprises at least one lipid and further comprising 0.1 to 1% by weight of at least one cellulose derivative.

58. The shaver as claimed in claim 57, wherein the at least one cellulose derivative includes at least one selected from the group consisting of hydroxymethylcelluloses and sodium carboxymethylcellulose.

59. The shaver as claimed in claim 57, wherein the at least one cellulose derivative includes hydroxyethyl-cellulose.
